(19) United States
(12) Patent Application Publication
(10) Pub. No.: US 2023/0098305 A1
(43) Pub. Date: Mar. 30, 2023

(54) SYSTEMS AND METHODS TO PRODUCE TISSUE IMAGING BIOMARKERS

(71) Applicants: HOLOGIC, INC., Marlborough, MA (US); SUPERSONIC IMAGINE, Aix-en-Provence (FR)

(72) Inventors: Shawn ST. PIERRE, Marlborough, MA (US); Christophe FRASCHINI, Aix-en-Provence (FR)

(21) Appl. No.: 17/910,144

(22) PCT Filed: Mar. 24, 2021
(86) PCT No.: PCT/US2021/023904

§ 371 Date: Sep. 8, 2022

Related U.S. Application Data
(60) Provisional application No. 63/000,707, filed on Mar. 27, 2020.

(51) Int. Cl. A61B 8/08; A61B 6/02; A61B 6/00; A61B 8/00

(52) U.S. Cl. CPC A61B 8/0825; A61B 8/485; A61B 8/5261; A61B 6/025; A61B 6/502; A61B 8/469; A61B 8/5223; A61B 8/488; A61B 8/5246; A61B 8/4263; A61B 5/055

(57) ABSTRACT

Systems and methods for mapping a region of interest within breast tissue utilize multiple layers of information to produce a unique digital fingerprint of breast tissue. X-ray and ultrasound imaging is combined with elastography and Doppler to create an architectural map of a breast including coordinates to mark one or more regions of interest. The architectural map can be utilized during future imaging procedures and surgeries to automatically and virtually indicate the location of previously biopsied lesions. The architectural map can be displayed on a user interface of a computing device to guide a user to the region of interest during imaging.

This application is being filed on Mar. 24, 2021, as a PCT International Patent Application and claims the benefit of priority to U.S. Provisional patent application Ser. No. 63/000,707, filed Mar. 27, 2020, the entire disclosure of which is incorporated by reference in its entirety.

### BACKGROUND

Medical imaging provides a non-invasive method to visualize the internal structure of a patient. Visualization methods can be used to screen for and diagnose cancer in a patient. For example, early screening can detect lesions within a breast that might be cancerous so that treatment can take place at an early stage in the disease.

Mammography and digital breast tomosynthesis (DBT) utilize x-ray radiation to visualize breast tissue. These techniques are often used to screen patients for potentially cancerous lesions. Traditional mammograms involve acquiring two-dimensional images of the breast from various angles. Tomosynthesis produces a plurality of x-ray images, each of discrete layers or slices of the breast, through the entire thickness thereof. Tomosynthesis pieces together a three-dimensional visualization of the breast from the two-dimensional images.

If a lesion is found, a diagnostic ultrasound is often the next step in determining whether the patient has a tumor. Ultrasound uses sound waves, typically produced by piezoelectric transducers, to image tissue in a patient. Ultrasound imaging provides a different view of tissue that can make it easier to identify solid masses. An ultrasound probe focuses the sound waves by producing an arc-shaped sound wave that travels into the body and is partially reflected from the layers between different tissues in the patient. The reflected sound wave is detected by the transducers and converted into electrical signals that can be processed by the ultrasound scanner to form an ultrasound image of the tissue.

After a lesion is identified during imaging, it may be determined that a biopsy should be performed on the tissue within the lesion. In places such as the United States, it is typical for healthcare professionals such as radiologists or surgeons to remove a small portion of tissue from the lesion and then mark the location for future reference. Markers are embedded in the tissue and can be composed of various materials such as titanium, ceramic, and nitinol. The markers are useful for identifying previously biopsied sites during future imaging procedures. Additionally, should the biopsy reveal that surgery should be performed to remove the lesion, the marker enables surgeons to easily identify the lesion's location.

While the markers are made of materials that are not harmful to the patient, there are problems associated with leaving foreign bodies in the patient. Approximately 85% of biopsies find that lesions are benign. Some markers can be felt by the patient— particularly on superficial lesions. These markers provide the patient with an unpleasant reminder of a stressful medical experience, whether or not the biopsy resulted in a cancer diagnosis. Additionally, about 15% of markers cannot be located by surgeons for follow up surgery. Therefore, a more effective method of marking biopsy sites is needed.

It is against this background that the present disclosure is made. Techniques and improvements are provided herein.

### SUMMARY

Examples of the disclosure are directed to a method of mapping a region of interest within a breast.

In one aspect, a method of mapping a target site within a breast includes capturing diagnostic medical images of breast tissue including the target site within the breast. Vascularity and stiffness of the breast tissue including the target site is measured. The images, vascularity, and stiffness are saved in an architectural map in an electronic record. In some examples, spectral parameters of the breast tissue are recorded and saved with the architectural map. In some examples, the architectural map of the breast tissue includes a margin of normal tissue around the target site.

In another aspect, a system for mapping a region of interest within a breast includes at least one data store; a processing device; and a memory storing instructions that, when executed by the processor, facilitate performance of operations. The operations include mapping the region of interest within the breast by recording at least one image of the region of interest using diagnostic medical imaging; measuring vascularity of the region of interest; and measuring density of the region of interest. The operations further include saving the at least one image, the vascularity, and the density as an architectural map in an electronic record associated with the breast.

In yet another aspect, a non-transitory machine-readable storage medium stores executable instructions that, when executed by a processor, facilitate performance of operations. The operations include: capturing ultrasound images of an entire breast; recording location coordinates of a region of interest within the breast; measuring, using microflow Doppler, vascularity of the breast tissue including the region of interest; measuring, using shear-wave elastography, stiffness of the breast tissue including the region of interest; and saving the images, the location coordinates, the vascularity, and the stiffness as an architectural map in an electronic record associated with the breast. The operations further include, at a later time, receiving scan information obtained from imaging of the breast; accessing the architectural map from the electronic record associated with the breast; and analyzing the scan information to identify the region of interest based on the architectural map.

The details of one or more techniques are set forth in the accompanying drawings and the description below. Other features, objects, and advantages of these techniques will be apparent from the description, drawings, and claims.

### BRIEF DESCRIPTION OF THE DRAWINGS

FIG. 1 illustrates an example system for mapping a region of interest within a breast.

FIG. 2 illustrates a schematic diagram of an example system for managing healthcare data including imaging data.

FIG. 3 is a schematic view of an exemplary x-ray imaging system.

FIG. 4 is a perspective view of the x-ray imaging system of FIG. 3.

FIG. 5 depicts the x-ray imaging system in a breast positioning state for left mediolateral oblique (LMLO) imaging orientation.

FIG. 6 depicts an example ultrasound imaging system.

FIG. 7 depicts an example of the ultrasound imaging system of FIG. 6 in use with a breast of a patient.

FIG. 8 illustrates a schematic of the different layers of information used to produce an architectural map of a breast.

FIG. 9 is a flow diagram illustrating an example method of mapping a region of interest within a breast.

FIG. 10 illustrates an example display of the graphical user interface of FIG. 1.

FIG. 11 is a schematic diagram of an exemplary computing system usable to implement one or more aspects of the present disclosure.

### DETAILED DESCRIPTION

The present disclosure is directed to systems and methods for mapping a region of interest within breast tissue. In particular, a unique digital fingerprint of breast tissue is created by combining various imaging techniques. During an ultrasound, a region of interest can be marked by making an architectural map of the area from two or more of 2D slides, elastography, textural analysis, quantitative ultrasound, and spectral parametric maps. The architectural map can be utilized during future imaging procedures and surgeries to automatically and virtually indicate the location of the region of interest. The architectural map can be displayed on a user interface of a computing device to guide a user to the region of interest during imaging. In some examples, the region of interest is a biopsied site. The biopsied site could be marked with a physical marker or not.

In some examples, two or more sets of imaging data are combined to provide a unique “fingerprint” of breast tissue including a region of interest. One set of a data is a top down image of the breast (from chest wall to skin). The top down image can be one or both of x-ray imaging and ultrasound imaging. The second set of data is navigation coordinates to focus in on the location of a region of interest. This could be pointing to a particular lesion or biopsy location within the breast tissue. These coordinates could include a “clock position” from a nipple, a depth within the breast, and a distance from the nipple. The third set of data provides a detailed view of the tissue immediately surrounding the region of interest. This is imaged with ultrasound technologies to measure vascularity and stiffness of the tissue. The vascularity can be measured with microflow Doppler to visualize a pattern of blood flow around a lesion. The stiffness, or density, can be measured with SHEARWAVE™ elastography, which outputs a color map representing relative values of stiffness throughout the tissue volume. Elastography can also be used to determine if a patient needs a follow up imaging exam based on finding microcalcifications in the breast tissue. In some examples, quantitative ultrasound imaging techniques can be used to provide additional information about the breast tissue. The resulting “fingerprint” or architectural map is used to help a healthcare professional navigate to a particular region of interest during a breast ultrasound.

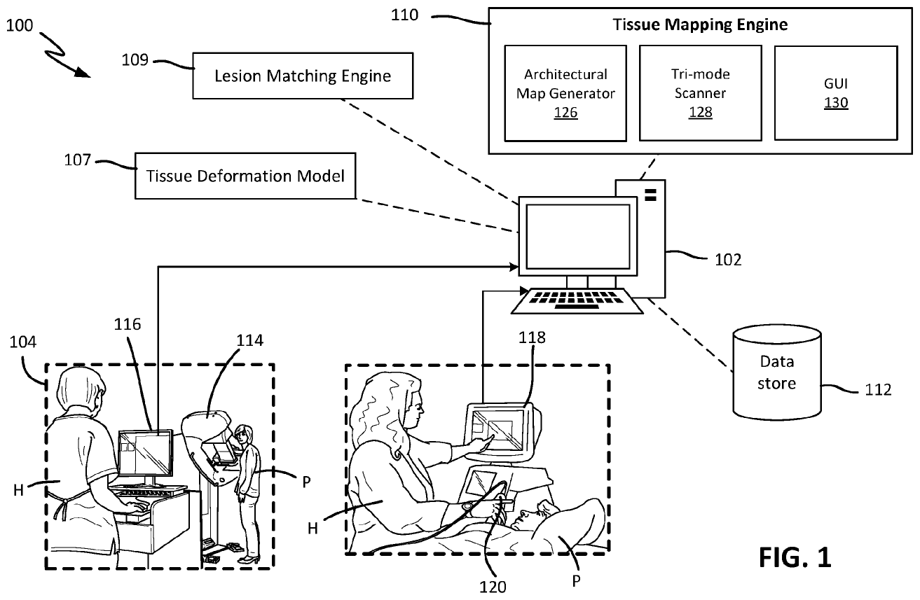

FIG. 1 illustrates an example tissue mapping system 100. In some examples, the tissue mapping system 100 operates to produce virtual biomarkers usable for imaging breast tissue. The system 100 includes a computing system 102, an x-ray imaging system 104, and an ultrasound imaging system 106. In some examples, the tissue mapping system 100 operates to produce a digital architectural map of breast tissue using information received from the x-ray imaging system 104 and ultrasound imaging system 106. The digital architectural map can be used to virtually mark regions of interest within a breast.

The computing system 102 operates to process and store information received from the x-ray imaging system 104 and ultrasound imaging system 106. In the example of FIG. 1, the computing system 102 includes a tissue mapping engine 110 and a data store 112. In some examples, the tissue mapping engine 110 and data store 112 are housed within the memory of the computing system 102. In some examples, the computing system 102 accesses the tissue mapping engine 110 and data store 112 from a remote server such as a cloud computing environment. In some examples, the computing system 102 also includes a tissue deformation model 107 and a lesion matching engine 109.

The tissue mapping engine 110 operates to create a digital architectural map of breast tissue by combining various types of information to produce imaging biomarkers of a region of interest. In some examples, the tissue mapping engine 110 includes an architectural map generator 126, a tri-mode scanner 128, and a graphical user interface (GUI) 130.

In some examples, the tissue mapping engine 110 receives, analyzes, and synthesizes data from elastography, Doppler, x-ray, and ultrasound to produce a digital architectural map of a region of interest within a breast. The architectural map generator 126 synthesizes together three layers of information to digitally mark a location of a location of interest within the breast.

One layer of information is an annotation of the location of the region of interest (ROI). The region of interest could be where a lesion has been identified as being potentially cancerous. The lesion could have been biopsied and a healthcare provider H wants to marks its location for future reference. In these examples, the ROI is the point at which the needle was inserted in the tissue for the biopsy. A physical marker may or may not be placed at the biopsy site in addition to recording an annotation of the location of the ROI. In other examples, the ROI is a lesion identified during tomography. In some examples, the annotation is a set of coordinates describing the location of the region of interest within a breast. In some examples, the coordinates include a clock position relative to a nipple, a depth from the surface of the breast, and a distance from the nipple. In some examples, the coordinates can be determined using the tissue deformation model 107, as described below.

Another layer of information is images of the entire breast. One or more of x-ray images and ultrasound images are taken of the breast to image all tissue from chest wall to skin surface. This provides a full, zoomed out view of the region of interest within the breast. In some examples, the images are taken using B mode ultrasound. In some examples, the images are taken using tomosynthesis. More than one type of imaging can be used together.

The third layer of information is vascularity and stiffness patterns of the region of interest and surrounding tissue. Vascularity can be measured using ultrasonography techniques such as Doppler. Ultrasound imaging can depict blood flow by detecting high-frequency sound waves bouncing of red blood cells to create images of blood vessels. In some examples, microflow Doppler imaging is employed to determine a pattern of vessels in the breast tissue including and surrounding the region of interest.

Stiffness of breast tissue can be determined with elastography measurements. Typically, ultrasound or magnetic resonance imaging techniques are utilized to visualize the relative stiffness of tissue. Quasistatic elastography compares ultrasound images of tissue before and after applying external pressure to the tissue. The portions of tissue that are deformed the least under pressure have the greatest stiffness. Acoustic radiation force impulse imaging (ARFI) and shear-wave elasticity imaging (SWEI) utilize acoustic radiation force to push tissue. ARFI measures how much tissue moves in response to pushes in multiple places to form a 2-D map of qualitative stiffness. SWEI measures how quickly waves propagate from where the tissue is pushed to various lateral positions to infer stiffness of the intervening tissue. ShearWave PLUS technology, developed by SuperSonic Imagine, is another method of measuring tissue stiffness that provides a quantitative, real-time two-dimensional map. ShearWave™ PLUS utilizes the same concepts as SWEI, but implements them with shear waves that move at supersonic speed and ultrafast imaging is used to register the propagation of the waves.

Magnetic resonance elastography (MRE) uses a mechanical vibrator on the surface of the patient's body to create shear waves that travel into deeper tissues. The velocity of the waves is measured with magnetic resonance imaging (MRI) and used to infer tissue stiffness (shear modulus). MRE produces a quantitative 3-D map of tissue stiffness as well as a conventional three-dimensional MRI image. Other methods of determining tissue stiffness are possible.

Optional fourth or fifth layers include additional information about the breast tissue. This information could be obtained using quantitative ultrasound techniques to obtain spectral parameters of the tissue.

The tissue deformation model 107 is utilized to analyze information obtained about a region of interest in a breast during x-ray imaging and convert that information into location identifying information usable to navigate to the same region of interest during ultrasound imaging. Coordinates of the region of interest determined during a mammogram while the breast tissue is under compression are converted into a different set of coordinates that are used to locate the region of interest during ultrasound imaging of the breast tissue not under compression. The tissue deformation model 107 predicts the ultrasound coordinates based on the density and volume of the breast being imaged.

In some examples, the tissue deformation model 107 is based on a deformation curve determined based on the amount of spread of the breast tissue this is recorded on a mammography paddle. With the known compression force of the paddle and the density determined by the x-ray, the tissue deformation model 107 is calculated for each patient.

The lesion matching engine 109 operates to analyze x-ray images of a target lesion and ultrasound images of a potential lesion to determine if the potential lesion is the same as the target lesion. An artificial intelligence system is trained on DBT cases confirmed with ultrasound to generate an image classifier. Images are analyzed using the classifier to compare lesions based on features such as shape, color, margins, orientation, texture, pattern, size, and depth within the breast. A confidence score can be generated indicating the likelihood that a lesion identified in ultrasound is the same lesion identified with tomosynthesis.

The data store 112 operates to store information received from the x-ray imaging system 104, ultrasound imaging system 106, and tissue mapping engine 110. In some examples, the data store 112 is actually two or more separate data stores. For example, one data store could be a remote data store that stores images from x-ray imaging systems. Another data store could be housed locally within the computing system 102. In some examples, the data store 112 could be part of an electronic medical record (EMR) system.

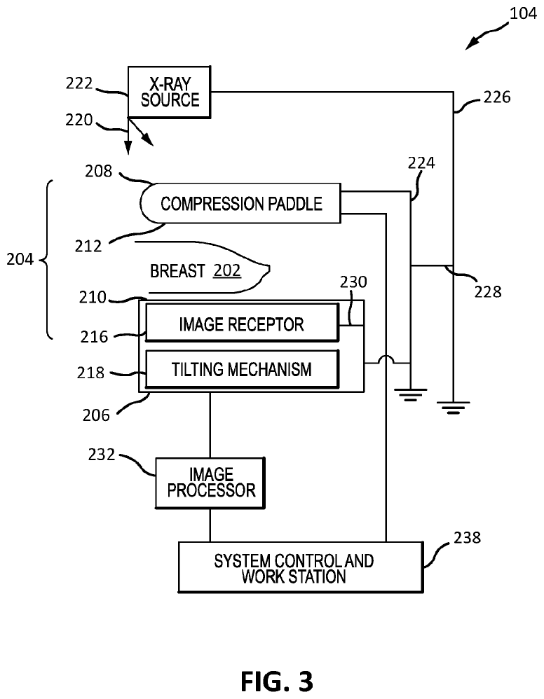

The x-ray imaging system 104 operates to take images of breast tissue using x-ray radiation. The x-ray imaging system 104 includes an x-ray imaging device 114 and an x-ray computing device 116 in communication with the x-ray imaging device 114. In some examples, the x-ray imaging system 104 performs tomosynthesis. The x-ray imaging device 114 is described in further detail in relation to FIGS. 3-5. The x-ray computing device 116 operates to receive inputs from a healthcare provider H to operate the x-ray imaging device 114 and view images received from the x-ray imaging device 114.

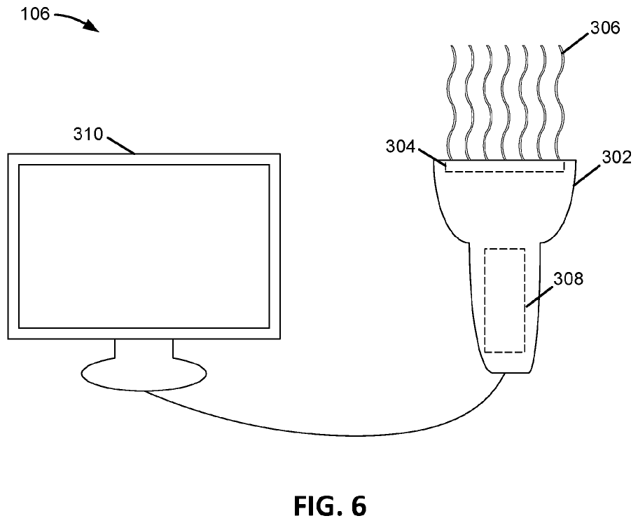

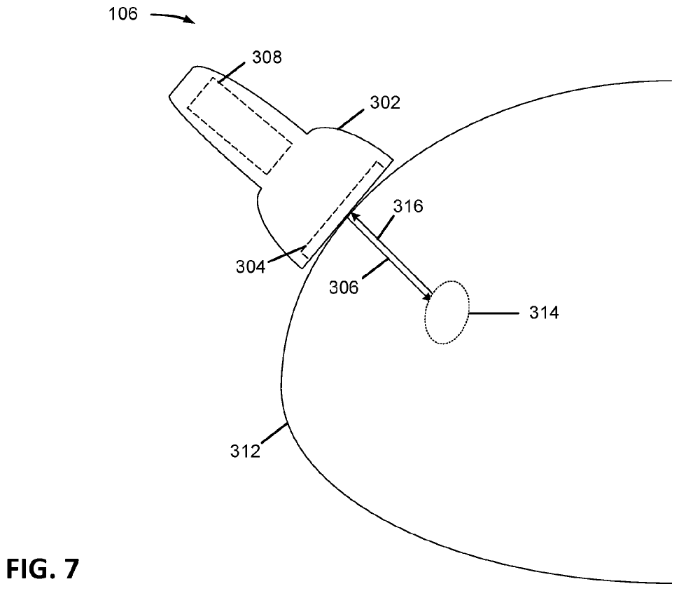

The ultrasound imaging system 106 operates to take images of breast tissue using ultrasonic sound waves. In some examples, the ultrasound imaging system 106 operates to perform one or more of elastography, textural analysis, and Doppler. The ultrasound imaging system 106 is described in further detail in relation to FIGS. 6-7. The ultrasound imaging system 106 includes an ultrasound computing device 118 and an ultrasound imaging device 120. The ultrasound computing device 118 operates to receive inputs from a healthcare provider H to operate the ultrasound imaging device 120 and view images received from the ultrasound imaging device 120.

FIG. 1 illustrates how information obtained from an x-ray imaging system 104 and an ultrasound imaging system 106 could be utilized by a tissue mapping engine 110 operating on a computing system 102. A healthcare provider H operates the x-ray computing device 116 to capture x-ray images of the breast of a patient P using the x-ray imaging device 114. The x-ray image may be taken as part of a routine health screening. During the screening, the healthcare provider H identifies one or more regions of interest in the patient P's breast that require additional analysis to determine if lesions within those regions of interest are potentially cancerous and require a biopsy. Healthcare providers H may include, for example, surgeons, doctors, nurses, technologists, and sonographers.

In some examples, coordinates for the regions of interest can be recorded at the x-ray computing device 116 and communicated to the computing system 102. The coordinates recorded by the x-ray computing device 116 are analyzed using a tissue deformation model 107. In some examples, a first set of coordinates identifies a location of a lesion identified while the breast is under compression. The first set of coordinates are translated into a second set of coordinates identifying a predicted location of the identified lesion while the breast is not under compression. A region of interest in the ultrasound image is identified that corresponds to the second set of coordinates. This enables a technician to identify the potential lesion in the ultrasound image.

In some examples, the x-ray images are displayed on a user interface of the ultrasound computing device 118 along with ultrasound images that are received from the ultrasound imaging device 120. In some examples, a visual marker is displayed on the image indicating the location of a target lesion. The healthcare provider H operating the ultrasound computing device 118 locates a potential lesion in an ultrasound image that is potentially a match for a lesion previously identified in an x-ray image for the same patient P. The ultrasound image and an indication of the potential lesion are communicated to the computing system 102 for analysis. In some examples, a mammography image, a target region of interest, and B-mode imaging is displayed on the same GUI. The GUI 130 helps to visually guide an operator of an ultrasound system to the region of interest while also automating documentation of an ultrasound probe's position, orientation, and annotations.

In some examples, x-ray images including an identified lesion and ultrasound images including a potential lesion are analyzed by the lesion matching engine 109 of the computing system 102. The lesion matching engine 109 outputs a confidence level indicator for the potential lesion and communicates that confidence level indicator to the ultrasound computing device 118. The confidence level indicator could be a numeric value, a color, or a category that is displayed on a GUI on the ultrasound computing device 118.

During the imaging procedure, a healthcare professional indicates one or more regions of interest to be recorded with a patient's record. These regions of interest could be lesions that were biopsied during the procedure. In some examples, the regions of interest are lesions identified for further observation. The marked location coordinates are determined for each region of interest.

The images and measurements generated by the x-ray imaging system 104 and ultrasound imaging system 106 are received at the tissue mapping engine 110. The tissue mapping engine 110 generates architectural maps of a patient's breast including at least one region of interest and saves the maps to the data store 112.

Later, when the patient returns for another examination or surgery, a healthcare provider may want to find a previously identified lesion or other region of interest within the patient's breast. A separate imaging procedure is performed after minutes, hours, days, or weeks have gone by. In some instances, the same patient could be examined with two different imaging procedures during the same visit to a healthcare facility. In some examples, the healthcare professional H may be interested in determining if there are any overall changes to the composition of the breast tissue. For example, the patient may have undergone chemotherapy and/or radiation since the last time the patient's breast tissue was imaged and the healthcare provider wants to see how treatment has affected the health of the breast tissue.

In one example, the healthcare professional H could be operating an ultrasound computing device 118 to image the patient P's breast. The ultrasound computing device 118 accesses the architectural map associated with the patient P from the data store 112. The tissue mapping engine 110 generates a GUI 130 to display on the ultrasound computing device 118. The tri-mode scanner 128 presents a tri-mode view of the breast that includes B-mode ultrasound images, vascularity, and stiffness. The tri-mode scanner reads the position of an ultrasound probe relative to the patient's breast and aids in navigation to the location coordinates of the previously identified region of interest.

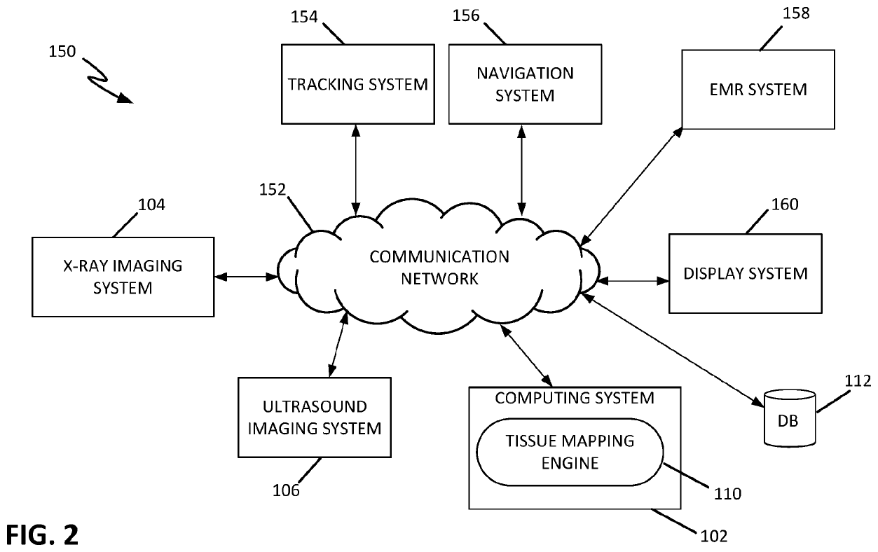

FIG. 2 illustrates a schematic diagram of an example system 150 for managing healthcare data including imaging data. The system 150 includes multiple computing components in communication with one another through a communication network 152. The computing components can include a tracking system 154, a navigation system 156, an EMR system 158, and a display system 160 in addition to the computing system 102, x-ray imaging system 104, ultrasound imaging system 106, and data store 112 described in FIG. 1.

It should be noted that, although the ‘systems’ are shown in FIG. 1 as functional blocks, different systems may be integrated into a common device, and the communication link may be coupled between fewer than all of the systems; for example, the tracking system 154, navigation system 156 and display system 160 may be included in an acquisition work station or a technologist work station which may control the acquisition of the images in a radiology suite. Alternatively, the navigation system 156 and tracking system 154 may be integrated into the ultrasound imaging system 106, or provided as standalone modules with separate communication links to the display 160, x-ray imaging system 104 and ultrasound system 106. Similarly, skilled persons will additionally appreciate that communication network 152 can be a local area network, wide area network, wireless network, internet, intranet, or other similar communication network.

In one example, the x-ray imaging system 104 is a tomosynthesis acquisition system which captures a set of projection images of a patient's breast as an x-ray tube scans across a path over the breast. The set of projection images is subsequently reconstructed to a three-dimensional volume which may be viewed as slices along any plane. The three-dimensional volume may be stored locally at the x-ray imaging system 104 (either on the x-ray imaging device 114 or on the x-ray computing device 116) or at a data store such as the data store 112 in communication with the x-ray imaging system 104 through the communication network 152. In some examples, the three-dimensional volume could be stored in a patient's file within an electronic medical record (EMR) system 158. Additional details regarding an example x-ray imaging system are described in reference to FIGS. 3-5.

The x-ray imaging system 104 may transmit the three-dimensional x-ray image volume to a navigation system 156 via the communication network 152, where such x-ray image can be stored and viewed. The navigation system 156 displays the x-ray image obtained by the x-ray imaging system. Once reconstructed for display on navigation system 156 the x-ray image can be reformatted and repositioned to view the image at any plane and any slice position or orientation. In some examples, the navigation system 156 displays multiple frames or windows on the same screen showing alternative positions or orientations of the x-ray-image slice.

Skilled persons will understand that the x-ray image volume obtained by x-ray imaging system 104 can be transmitted to navigation system 156 at any point in time and is not necessarily transmitted immediately after obtaining the x-ray image volume, but instead can be transmitted on the request of navigation system 156. In alternative examples, the x-ray image volume is transmitted to navigation system 156 by a transportable media device, such as a flash drive, CD-ROM, DVD-ROM, diskette, or other such transportable media device.

The ultrasound imaging system 106 obtains an ultrasound image of a tissue of a patient, typically using an ultrasound probe, which is used to image a portion of a tissue of a patient within the field of view of the ultrasound probe. For instance, the ultrasound imaging system 106 may be used to image a breast. The ultrasound imaging system 106 obtains and displays an ultrasound image of a patient's anatomy within the field of view of the ultrasound probe and typically displays the image in real-time as the patient is being imaged. In some examples, the ultrasound image can additionally be stored on a storage medium, such as a hard drive, DVD-ROM, flash drive or diskette, for reconstruction or playback at a later time. Additional details regarding the ultrasound imaging system are described in reference to FIGS. 6-7.

In some examples, the navigation system 156 can access the ultrasound image, and in such examples the ultrasound imaging system 106 is further connected to the communication network 152 and a copy of the ultrasound image obtained by the ultrasound imaging system 106 can be transmitted to the navigation system 156 via communication network 152. In other examples, the navigation system 156 can remotely access and copy the ultrasound image via the communication network 152. In alternative examples, a copy of the ultrasound image can be stored on the data store 112 or EMR system 158 in communication with the navigation system 156 via the communication network 152 and accessed remotely by the navigation system 156.

The tracking system 154 is in communication with the navigation system 156 via the communications network 152 and may track the physical position in which the ultrasound imaging system 106 is imaging the tissue of the patient. In some examples, the tracking system 154 can be connected directly to the navigation system 156 via a direct communication link or wireless communication link. The tracking system 154 tracks the position of transmitters connected to ultrasound imaging system 106 and provides the navigation system 156 with data representing their coordinates in a tracker coordinate space. In some examples, the tracking system 154 may be an optical tracking system comprising an optical camera and optical transmitters, however skilled persons will understand that any device or system capable of tracking the position of an object in space can be used. For example, skilled persons will understand that in some examples a radio frequency (RF) tracking system can be used, comprising an RF receiver and RF transmitters.

The ultrasound imaging system 106 may be configured for use with the navigation system 156 by a calibration process using the tracking system 154. Transmitters that are connected to the ultrasound probe of ultrasound imaging system 106 may transmit their position to tracking system 154 in the tracker coordinate space, which in turn provides this information to navigation system 156. For example, transmitters may be positioned on the probe of the ultrasound imaging system 106 so that the tracking system 154 can monitor the position and orientation of the ultrasound probe and provide this information to the navigation system 156 in the tracker coordinate space. The navigation system 156 may use this tracked position to determine the position and orientation of the ultrasound probe, relative to the tracked position of the transmitters.

In some examples, configuration occurs using a configuration tool. In such examples, the position and orientation of the configuration tool may be additionally tracked by tracking system 154. During configuration the configuration tool contacts the transducer face of the ultrasound probe of the ultrasound imaging system 106 and the tracking system 154 transmits information representing the position and orientation of the configuration tool in the tracker coordinate space to the navigation system 156. The navigation system 156 may determine a configuration matrix that can be used to determine the position and orientation of the field of view of the ultrasound probe in the tracker coordinate space, based on the tracked position of the transmitters connected to the ultrasound probe. In alternative examples, a database having configuration data of a plurality of brands or models of various ultrasound probes can be used to pre-load a field of view configuration into the navigation system 156 during configuration.

Once the ultrasound imaging system 106 is configured with the navigation system 156, the tissue of a patient can be imaged with ultrasound imaging system 106. During ultrasound imaging, the tracking system 154 monitors the position and orientation of the ultrasound probe of the ultrasound imaging system 106 and provides this information in the tracker coordinate space to the navigation system 156. Since the ultrasound imaging system 106 has been configured for use with the navigation system 156, the navigation system 156 is able to determine position and orientation of the field of view of the ultrasound probe of the ultrasound imaging system 106.

The navigation system 156 can be configured to co-register an ultrasound image with an x-ray image. In some examples, the navigation system 156 can be configured to transform the position and orientation of the field of view of the ultrasound probe from the tracker coordinate space to a position and orientation in the x-ray image, for example, to x-ray system coordinates. This can be accomplished by tracking the position and orientation of the ultrasound probe and transmitting this positional information in the tracker coordinate space to navigation system 156 and relating this positional information to the x-ray coordinate system. In some examples, the co-registered images are displayed on the GUI 130.

For example, a user can select an anatomical plane within the x-ray image, and the user can then manipulate the position and orientation of a tracked ultrasound probe to align the field of view of the ultrasound probe with the selected anatomical plane. Once alignment is achieved, the associated tracker space coordinates of the ultrasound image can be captured. Registration of the anatomic axes (superior-inferior (SI), left-right (LR) and anterior-posterior (AP)) between the x-ray image and the tracker coordinate space can be determined from the relative rotational differences between the tracked ultrasound field of view orientation and the selected anatomical plane using techniques known to those of skill in the art.

This configuration may further include the selection of landmarks within the x-ray image, for example, using an interface permitting a user to select an anatomical target. In some examples, the landmark can be an internal tissue landmark, such as veins or arteries, and in other examples, the landmark can be an external landmark, such as a fiducial skin marker or external landmark, such as a nipple. The same landmark selected in the x-ray image can be located with the ultrasound probe, and upon location, a mechanism can be provided for capturing coordinates of the representation of the target in the tracker coordinate space. The relative differences between the coordinates of the target in the x-ray image and the coordinates of the target in the tracker coordinate space are used to determine the translational parameters required to align the two co-ordinate spaces. The plane orientation information acquired previously can be combined with the translation parameters to provide a complete 4×4 transformation matrix capable of co-registering the two coordinate spaces.

The navigation system 156 can then use the transformation matrix to reformat the x-ray image being displayed so that the slice of tissue being displayed is in the same plane and in the same orientation as the field of view of the ultrasound probe of the ultrasound imaging system 106. Matched ultrasound and x-ray images may then be displayed side by side, or directly overlaid in a single image viewing frame. In some examples, the navigation system 156 can display additional x-ray images in separate frames or positions on a display screen. For example, the x-ray image can be displayed with a graphical representation of the field of view of the ultrasound imaging system 106 wherein the graphical representation of the field of view is shown slicing through a 3D representation of the x-ray image. In other examples annotations can be additionally displayed, these annotations representing, for example, the position of instruments imaged by the ultrasound imaging system 106, such as biopsy needles, guidance wires, imaging probes or other similar devices.

In other examples, the ultrasound image being displayed by the ultrasound imaging system 106 can be superimposed on the slice of the x-ray image being displayed by the navigation system 156 so that a user can view both the x-ray and ultrasound images simultaneously, overlaid on the same display. It is typically difficult to superimpose ultrasound images over x-ray images because compression and orientation of breasts is different depending on the technology used. The computing system 102 can modify the images based on analyses performed on the images to produce artificially combined images. In some examples, the navigation system 156 can enhance certain aspects of the super imposed ultrasound or x-ray images to increase the quality of the resulting combined image.

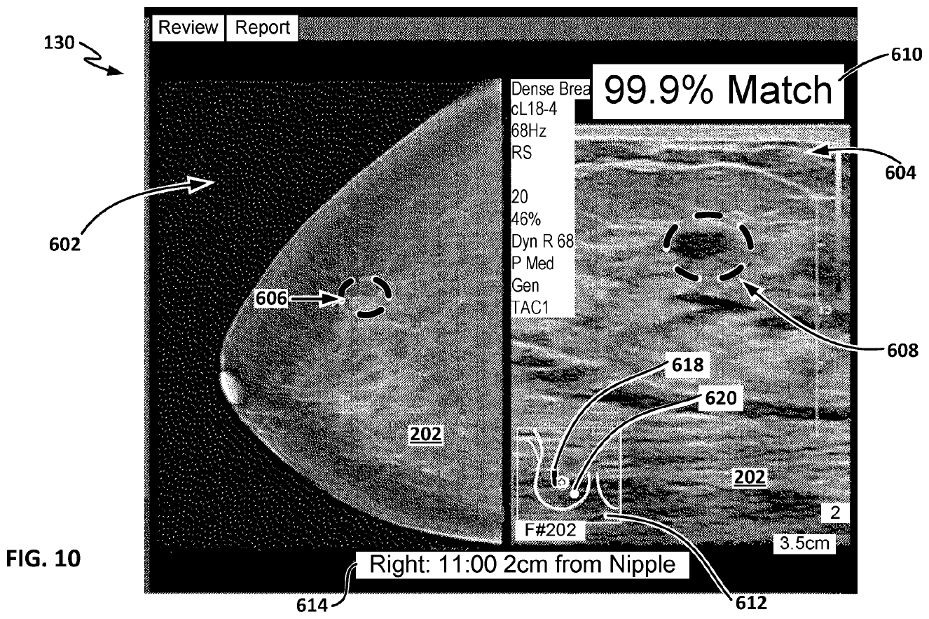

As described in FIG. 1, the computing system 102 operating a tissue mapping engine 110 combines information from x-ray images, ultrasound images, location coordinates, spectral parameters, vascularity measurements, and stiffness measurements to construct an architectural map of breast tissue. The map can be viewed using a display of the computing system 102 or computing devices in communication with the ultrasound imaging system 106 to locate a region of interest during imaging. In some examples, visualizations of vascularity and stiffness of tissue are overlaid B-mode ultrasound images to produce a combination view to aid in identifying a previously marked region of interest in a breast. In other examples, the combination view simply shows ultrasound images with an indication of the region of the interest and the current location of the ultrasound probe, as shown in the example of FIG. 10. X-ray images of the ROI can also be displayed next to the ultrasound images.

The electronic medical record system 158 stores a plurality of electronic medical records (EMRs). Each EMR contains the medical and treatment history of a patient. Examples of electronic medical records systems 158 include those developed and managed by Epic Systems Corporation, Cerner Corporation, Allscripts, and Medical Information Technology, Inc. (Meditech).

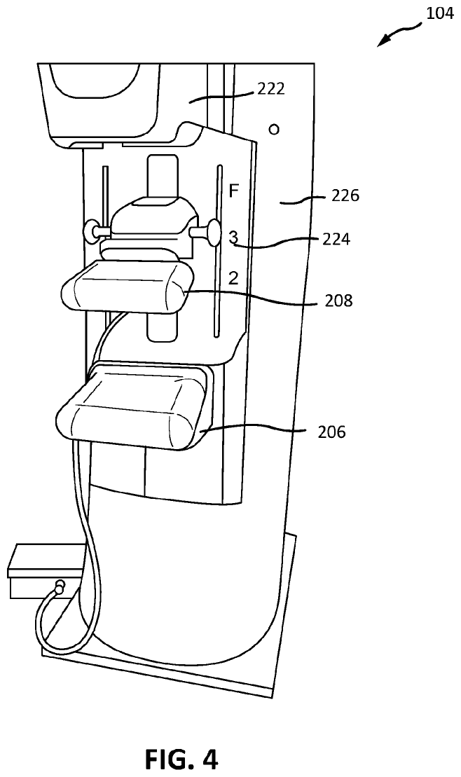

FIG. 3 is a schematic view of an exemplary x-ray imaging system 104. FIG. 4 is a perspective view of the x-ray imaging system 104. Referring concurrently to FIGS. 3 and 4, the x-ray imaging system 104 immobilizes a patient's breast 202 for x-ray imaging (either or both of mammography and tomosynthesis) via a breast compression immobilizer unit 204 that includes a static breast support platform 206 and a moveable compression paddle 208. The breast support platform 206 and the compression paddle 208 each have a compression surface 210 and 212, respectively, that move towards each other to compress and immobilize the breast 202. In known systems, the compression surface 210, 212 is exposed so as to directly contact the breast 202. The platform 206 also houses an image receptor 216 and, optionally, a tilting mechanism 218, and optionally an anti-scatter grid. The immobilizer unit 204 is in a path of an imaging beam 220 emanating from x-ray source 222, such that the beam 220 impinges on the image receptor 216.

The immobilizer unit 204 is supported on a first support arm 224 and the x-ray source 222 is supported on a second support arm 226. For mammography, support arms 224 and 226 can rotate as a unit about an axis 228 between different imaging orientations such as CC and MLO, so that the system 104 can take a mammogram projection image at each orientation. In operation, the image receptor 216 remains in place relative to the platform 206 while an image is taken. The immobilizer unit 204 releases the breast 202 for movement of arms 224, 226 to a different imaging orientation. For tomosynthesis, the support arm 224 stays in place, with the breast 202 immobilized and remaining in place, while at least the second support arm 226 rotates the x-ray source 222 relative to the immobilizer unit 204 and the compressed breast 202 about the axis 228. The system 104 takes plural tomosynthesis projection images of the breast 202 at respective angles of the beam 220 relative to the breast 202.

Concurrently and optionally, the image receptor 216 may be tilted relative to the breast support platform 206 and in sync with the rotation of the second support arm 226. The tilting can be through the same angle as the rotation of the x-ray source 222, but may also be through a different angle selected such that the beam 220 remains substantially in the same position on the image receptor 216 for each of the plural images. The tilting can be about an axis 230, which can but need not be in the image plane of the image receptor 216. The tilting mechanism 218 that is coupled to the image receptor 216 can drive the image receptor 216 in a tilting motion.

For tomosynthesis imaging and/or CT imaging, the breast support platform 206 can be horizontal or can be at an angle to the horizontal, e.g., at an orientation similar to that for conventional MLO imaging in mammography. The x-ray imaging system 104 can be solely a mammography system, a CT system, or solely a tomosynthesis system, or a “combo” system that can perform multiple forms of imaging. An example of such a combo system has been offered by the assignee hereof under the trade name Selenia Dimensions.

When the system is operated, the image receptor 216 produces imaging information in response to illumination by the imaging beam 220, and supplies it to an image processor 232 for processing and generating breast x-ray images. A system control and work station unit 238 including software controls the operation of the system and interacts with the operator to receive commands and deliver information including processed-ray images.

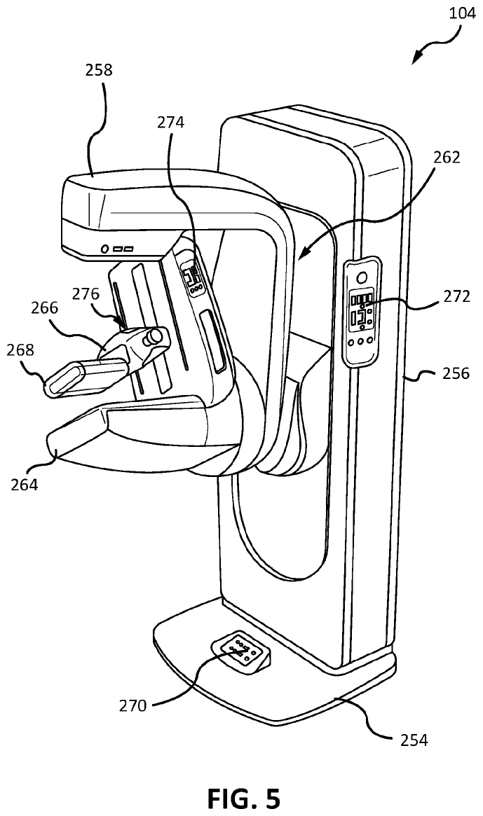

FIG. 5 depicts an exemplary x-ray imaging system 104 in a breast positioning state for left mediolateral oblique MLO (LMLO) imaging orientation. A tube head 258 of the system 104 is set in an orientation so as to be generally parallel to a gantry 256 of the system 104, or otherwise not normal to the flat portion of a support arm 260 against which the breast is placed. In this position, the technologist may more easily position the breast without having to duck or crouch below the tube head 258.

The x-ray imaging system 104 includes a floor mount or base 254 for supporting the x-ray imaging system 104 on a floor. The gantry 256 extends upwards from the floor mount 252 and rotatably supports both the tube head 258 and a support arm 260. The tube head 258 and support arm 260 are configured to rotate discretely from each other and may also be raised and lowered along a face 262 of the gantry so as to accommodate patients of different heights. An x-ray source, described elsewhere herein and not shown here, is disposed within the tube head 258. The support arm 260 includes a support platform 264 that includes therein an x-ray receptor and other components (not shown). A compression arm 266 extends from the support arm 260 and is configured to raise and lower linearly (relative to the support arm 260) a compression paddle 268 for compression of a patient breast during imaging procedures. Together, the tube head 258 and support arm 260 may be referred to as a C-arm.

A number of interfaces and display screens are disposed on the x-ray imaging system 104. These include a foot display screen 270, a gantry interface 272, a support arm interface 274, and a compression arm interface 276. In general the various interfaces 272, 274, and 276 may include one or more tactile buttons, knobs, switches, as well as one or more display screens, including capacitive touch screens with graphic user interfaces (GUIs) so as to enable user interaction with and control of the x-ray imaging system 104. In examples, the interfaces 272, 274, 276 may include control functionality that may also be available on a system control and work station, such as the x-ray computing device 116 of FIG. 1. Any individual interface 272, 274, 276 may include functionality available on other interfaces 272, 274, 276, either continually or selectively, based at least in part on predetermined settings, user preferences, or operational requirements. In general, and as described below, the foot display screen 270 is primarily a display screen, though a capacitive touch screen might be utilized if required or desired.

In examples, the gantry interface 272 may enable functionality such as: selection of the imaging orientation, display of patient information, adjustment of the support arm elevation or support arm angles (tilt or rotation), safety features, etc. In examples, the support arm interface 274 may enable functionality such as adjustment of the support arm elevation or support arm angles (tilt or rotation), adjustment of the compression arm elevation, safety features, etc. In examples, the compression arm interface 276 may enable functionality such as adjustment of the compression arm elevation, safety features, etc. Further, one or more displays associated with the compression arm interface 276 may display more detailed information such as compression arm force applied, imaging orientation selected, patient information, support arm elevation or angle settings, etc. The foot display screen 270 may also display information such as displayed by the display(s) of the compression arm interface 276, or additional or different information, as required or desired for a particular application.

FIG. 6 depicts an example of an ultrasound imaging system 106. The ultrasound imaging system 106 includes an ultrasound probe 302 that includes an ultrasonic transducer 304. The ultrasonic transducer 304 is configured to emit an array of ultrasonic sound waves 306. The ultrasonic transducer 304 converts an electrical signal into ultrasonic sound waves 306. The ultrasonic transducer 304 may also be configured to detect ultrasonic sound waves, such as ultrasonic sound waves that have been reflected from internal portions of a patient, such as lesions within a breast. In some examples, the ultrasonic transducer 304 may incorporate a capacitive transducer and/or a piezoelectric transducer, as well as other suitable transducing technology.

The ultrasonic transducer 304 is also operatively connected (e.g., wired or wirelessly) to a display 310. The display 310 may be a part of a computing system, such as the ultrasound computing device 118 of FIG. 2, which includes processors and memory configured to produce and analyze ultrasound images. The display 310 is configured to display ultrasound images based on an ultrasound imaging of a patient.

The ultrasound imaging performed in the ultrasound imaging system 106 is primarily B-mode imaging, which results in a two-dimensional ultrasound image of a cross-section of a portion of the interior of a patient. The brightness of the pixels in the resultant image generally corresponds to amplitude or strength of the reflected ultrasound waves.

Other ultrasound imaging modes may also be utilized. For example, the ultrasound probe may operate in a 3D ultrasound mode that acquires ultrasound image data from a plurality of angles relative to the breast to build a 3D model of the breast.

In some examples, ultrasound images may not be displayed during the acquisition process. Rather, the ultrasound data is acquired and a 3D model of the breast is generated without B-mode images being displayed.

The ultrasound probe 302 may also include a probe localization transceiver 308. The probe localization transceiver 308 is a transceiver that emits a signal providing localization information for the ultrasound probe 302. The probe localization transceiver 308 may include a radio frequency identification (RFID) chip or device for sending and receiving information as well as accelerometers, gyroscopic devices, or other sensors that are able to provide orientation information. For instance, the signal emitted by the probe localization transceiver 308 may be processed to determine the orientation or location of the ultrasound probe 302. The orientation and location of the ultrasound probe 302 may be determined or provided in three-dimensional components, such as Cartesian coordinates or spherical coordinates. The orientation and location of the ultrasound probe 302 may also be determined or provided relative to other items, such as an incision instrument, a marker, a magnetic direction, a normal to gravity, etc. With the orientation and location of the ultrasound probe 302, additional information can be generated and provided to the surgeon to assist in guiding the surgeon to a lesion within the patient, as described further below. While the term transceiver is used herein, the term is intended to cover both transmitters, receivers, and transceivers, along with any combination thereof.

FIG. 7 depicts an example of the ultrasound imaging system 106 in use with a breast 312 of a patient. The ultrasound probe 302 is in contact with a portion of the breast 312. In the position depicted in FIG. 7, the ultrasound probe 302 is being used to image a lesion 314 of the breast 312. To image the lesion 314, the ultrasonic transducer 304 emits an array of ultrasonic sound waves 306 into the interior of the breast 312. A portion of the ultrasonic sound waves 306 are reflected off internal components of the breast, such as the lesion 314 when the lesion is in the field of view, and return to the ultrasound probe 302 as reflected ultrasonic sound waves 316. The reflected ultrasonic sound waves 316 may be detected by the ultrasonic transducer 304. For instance, the ultrasonic transducer 304 receives the reflected ultrasonic sound waves 316 and converts the reflected ultrasonic sound waves 316 into an electric signal that can be processed and analyzed to generate ultrasound image data on display 310.

The depth of the lesion 314 or other objects in an imaging plane may be determined from the time between a pulse of ultrasonic waves 306 being emitted from the ultrasound prove 302 and the reflected ultrasonic waves 316 being detected by the ultrasonic probe 302. For instance, the speed of sound is well-known and the effects of the speed of sound based on soft tissue are also determinable. Accordingly, based on the time of flight of the ultrasonic waves 306 (more specifically, half the time of flight), the depth of the object within an ultrasound image may be determined. Other corrections or methods for determining object depth, such as compensating for refraction and variant speed of waves through tissue, may also be implemented. Those having skill in the art will understand further details of depth measurements in medical ultrasound imaging technology. Such depth measurements and determinations may be used to build a 3D model of the breast 312. This 3D model can be useful for various other applications where the 3D model is correlated with other 3D breast schemes used by other types of technology.

In addition, multiple frequencies or modes of ultrasound techniques may be utilized. For instance, real time and concurrent transmit and receive multiplexing of localization frequencies as well as imaging frequencies and capture frequencies may be implemented. Utilization of these capabilities provide information to co-register or fuse multiple data sets from the ultrasound techniques to allow for visualization of lesions and other medical images on the display 310. The imaging frequencies and capture sequences may include B-mode imaging (with or without compounding), Doppler modes (e.g., color, duplex), harmonic mode, shearwave and other elastography modes, and contrast-enhanced ultrasound, among other imaging modes and techniques.

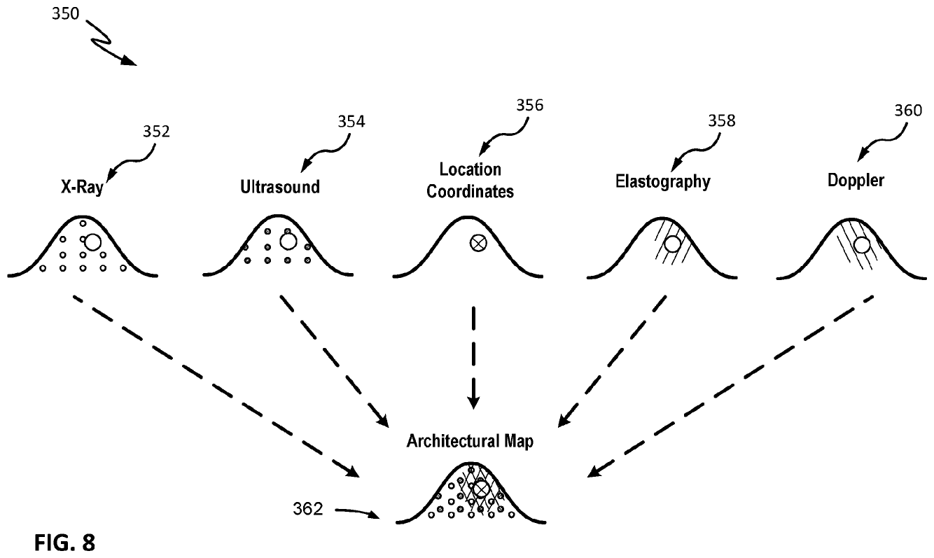

FIG. 8 shows a schematic diagram of the various layers of information that are used to build an architectural map of a breast. X-ray images 352 and ultrasound images 354 of the entire breast are taken and aligned over each other. The tissue deformation model 107 of FIG. 1 can be utilized to determine the coordinates 356 for the location of the region of interest. If the region of interest is a lesion, the lesion matching engine 109 can be used to confirm that the same lesion found in an ultrasound image is the one first identified in a mammogram. Elastography 358 and Doppler 360 are used to determine the stiffness and vascularity of the tissue surrounding the region of interest. Maps indicating relative stiffness and vascularity patterns are laid over the x-ray and ultrasound images of the breast to provide an overall view of the breast and region of interest in the resultant architectural map 362.

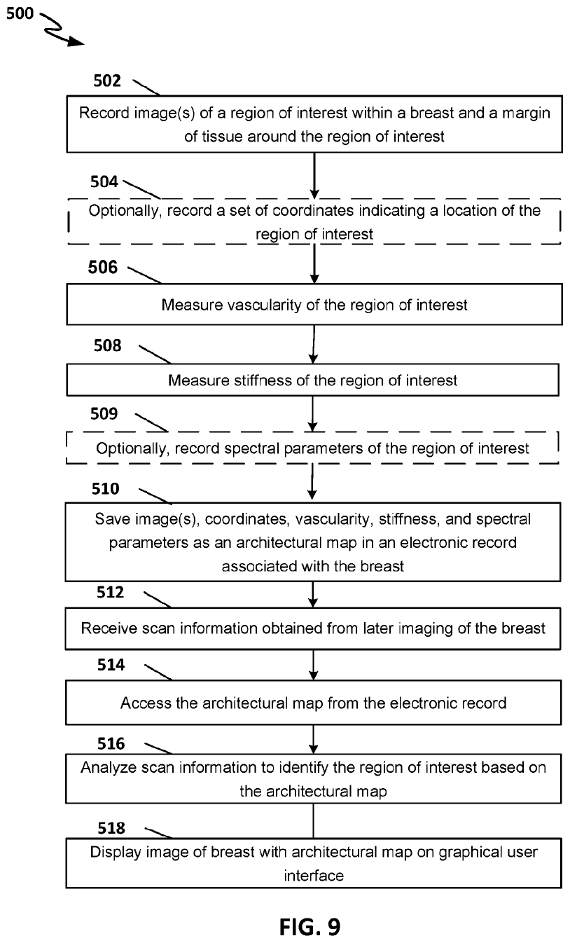

Referring now to FIG. 9, an example method 500 of mapping a region of interest within a breast is described. In some examples, the systems and devices described in FIGS. 1-7 are usable to implement the method 500. In particular, the computing system 102 of FIGS. 1-2 operates to implement the steps of the method 500 to generate an architectural map of a region of interest within a breast and aid a healthcare provider in later locating that region of interest during an imaging procedure or surgery.

At operation 502, images of a region of interest within a breast are recorded. The region of interest can be a lesion or an area of tissue including one or more lesions. In some examples, more than one region of interest is identified within a breast. In some examples, a margin of tissue around the region of interest is also imaged. The margin of tissue can vary in amount, but is intended to provide some “normal” tissue to compare the region of interest to. In some examples, images are taken of the entire breast—from chest wall to skin. The images can be recorded with one or both of x-ray and ultrasound imaging techniques.

At operation 504, a set of coordinates indicating a location of the region of interest can optionally be recorded. Input can be received from a healthcare professional indicating the region of interest on a computing device associated with an imaging device. Coordinates are generated for the location indicated by the healthcare professional. In some examples, the coordinates include a clock position relative to a nipple, a distance from the nipple, and a depth from the skin of the breast.

At operation 506, vascularity of the region of interest is measured. In some examples, vascularity is measured with an ultrasonography technique such as Doppler. In some examples, a margin of tissue around the region of interest is also measured to establish a baseline for normal tissue.

At operation 508, stiffness of the region of interest is measured. In some examples, stiffness is measured with an ultrasonography technique such as shear wave elasticity imaging. In some examples, a margin of tissue around the region of interest is also measured to establish a baseline for normal tissue.

At operation 509, spectral parameters of the region of interest are optionally recorded. Spectral parameters include quantitative ultrasound techniques such as spectral flow and scatter parameter.

At operation 510, the recorded images, vascularity measurements, and stiffness measurements are combined to generate an architectural map. In some examples, both ultrasound and x-ray imaging is used in combination to create the architectural map. In some examples, the architectural map further includes location coordinates. In some examples, spectral parameters are also included in the architectural map. The architectural map becomes more accurate and useful as more information is included. The architectural map is saved in an electronic record associated with the breast.

At operation 512, scan information from later imaging of the breast is received. Generally this could be ultrasound images that are taken in real time. The breast may be imaged at a later time for the purpose of performing surgery to remove a lesion. In some instances the breast may be imaged to determine if any changes have taken place in the tissue compared to a previous imaging. For example, a breast might be imaged to determine that the tissue surrounding a location where a biopsy was taken or a lesion was removed to determine if any changes have occurred indicating that cancerous tissue has begun to grow in that location. In some examples, the later imaging of the breast occurs at least an hour after the imaging of operation 502. In some examples, the later imaging occurs at least one day later. In some examples, the later imaging occurs at least one month later.

At operation 514, the architectural map of the breast is accessed from the electronic record. In some examples, the architectural map is accessed from a patient's record stored within an electronic medical record (EMR) system.

At operation 516, the scan information is analyzed to identify the region of interest based on the architectural map. In some examples, coordinates are used to guide an ultrasound probe to the region of interest. The coordinates can be used by a navigation system such as the navigation system 156 of FIG. 2 in conjunction with information from the tracking system 154 to guide a healthcare professional to the region of interest.

At operation 518, an image of the breast is displayed with information from the architectural map on a graphical user interface. In some examples, a combined view of the breast tissue is shown that includes x-ray and/or ultrasound images overlaid with an indication of the location of the region of interest. This combined view is effective for aiding a healthcare provider in identifying and navigating a region of interest within a breast. Additionally, the combined view is useful for identifying any changes that have occurred within the breast tissue. Such changes could affect the vascularity or stiffness of the tissue. Additionally, changes in density of the tissue can be identified with ultrasound or x-ray images that are included in the combined view. An example of this combined view is shown in FIG. 10.

FIG. 10 shows an example of the GUI 130 of FIG. 1. In some examples, the GUI 130 is displayed on a computing device such as the ultrasound computing device 118 of FIG. 1. In the example of FIG. 10, the GUI 130 displays an x-ray image 602 and an ultrasound image 604 of a breast 202 side-by-side. A target lesion 606 previously identified during x-ray imaging is indicated in the x-ray image 602 with a visual marker. The corresponding ultrasound image 604 of the breast 202 shows an indication of a potential lesion 608. A confidence level indicator 610 is displayed providing the likelihood that the target lesion 606 and potential lesion 608 are a match as a percentage. In this example, there is a 99.9% match.

The GUI 130 also includes a diagram 612 indicating the location on the breast 202 where the ultrasound image 604 is being taken. This diagram 612 includes a marker 620 for the ROI location as well as an indicator 618 of the current location of the ultrasound probe. Additionally, coordinates 614 are displayed. In this example, the coordinates 614 indicate the location of a potential lesion in the right breast at the 11:00 clock position, 2 cm from the nipple.

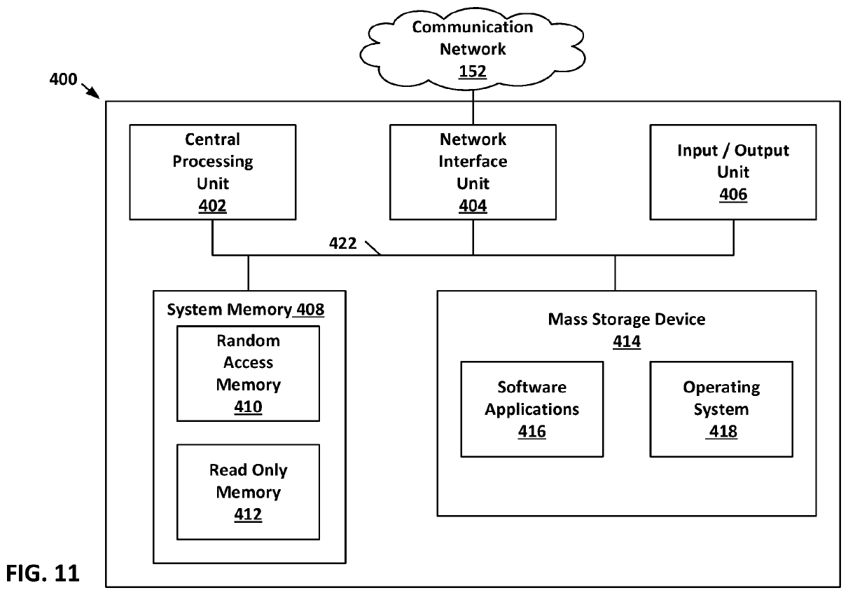

FIG. 11 is a block diagram illustrating an example of the physical components of a computing device 400. The computing device 400 could be any computing device utilized in conjunction with the tissue mapping system 100 or the system 150 for managing imaging data such as the computing system 102, x-ray computing device 116, and ultrasound computing device 118.

In the example shown in FIG. 11, the computing device 400 includes at least one central processing unit (“CPU”) 402, a system memory 408, and a system bus 422 that couples the system memory 408 to the CPU 402. The system memory 408 includes a random access memory (“RAM”) 410 and a read-only memory (“ROM”) 412. A basic input/output system that contains the basic routines that help to transfer information between elements within the computing device 400, such as during startup, is stored in the ROM 412. The computing system 400 further includes a mass storage device 414. The mass storage device 414 is able to store software instructions and data.

The mass storage device 414 is connected to the CPU 402 through a mass storage controller (not shown) connected to the system bus 422. The mass storage device 414 and its associated computer-readable storage media provide non-volatile, non-transitory data storage for the computing device 400. Although the description of computer-readable storage media contained herein refers to a mass storage device, such as a hard disk or solid state disk, it should be appreciated by those skilled in the art that computer-readable data storage media can include any available tangible, physical device or article of manufacture from which the CPU 402 can read data and/or instructions. In certain examples, the computer-readable storage media includes entirely non-transitory media.

Computer-readable storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the computing device 400.

According to some examples, the computing device 400 can operate in a networked environment using logical connections to remote network devices through a network 152, such as a wireless network, the Internet, or another type of network. The computing device 400 may connect to the network 152 through a network interface unit 404 connected to the system bus 422. It should be appreciated that the network interface unit 404 may also be utilized to connect to other types of networks and remote computing systems. The computing device 400 also includes an input/output controller 406 for receiving and processing input from a number of other devices, including a touch user interface display screen, or another type of input device. Similarly, the input/output controller 406 may provide output to a touch user interface display screen or other type of output device.

As mentioned briefly above, the mass storage device 414 and the RAM 410 of the computing device 400 can store software instructions and data. The software instructions include an operating system 418 suitable for controlling the operation of the computing device 400. The mass storage device 414 and/or the RAM 410 also store software instructions, that when executed by the CPU 402, cause the computing device 400 to provide the functionality discussed in this document.

The methods and systems described herein provide many efficiencies and advantages over existing solutions for imaging breast tissue. Synthesis of multiple imaging modalities with location coordinates provides greater accuracy in identifying lesions or other regions of interest within a breast that are identified by healthcare professionals during an imaging procedure. The system coordinates stiffness and elasticity maps with ultrasound and/or x-ray images to produce an information-rich view of the breast tissue. The healthcare professional does not need to access and view multiple different types of images to make an assessment of the tissue. By automatically combining these sets of information, fewer interactions with computing systems are required. In some examples, the tri-mode view of the breast is produced by automatically accessing the architectural map for a patient from the patient's EMR. Fewer requests for data are required and therefore, the functioning of the computing system is improved.

Although various embodiments and examples are described herein, those of ordinary skill in the art will understand that many modifications may be made thereto within the scope of the present disclosure. Accordingly, it is not intended that the scope of the disclosure in any way be limited by the examples provided.

What is claimed is:

1. A method of mapping a target site within a breast, the method comprising:
capturing diagnostic medical images of breast tissue including the target site within the breast;
measuring vascularity of the breast tissue including the target site;
measuring stiffness of the breast tissue including the target site; and
saving the images, vascularity, and stiffness as an architectural map of the target site in an electronic record.

2. The method of claim 1, further comprising recording spectral parameters of the breast tissue and saving the spectral parameters with the architectural map.

3. The method of claim 2, further comprising:
at a later time, receiving scan information obtained from imaging of the breast;
accessing the electronic record associated with the breast; and
analyzing the scan information to identify the target site based on the architectural map.

4. The method of claim 3, further comprising comparing the scan information with the architectural map in the electronic record to identify any changes in the breast tissue.

5. The method of claim 3, wherein the later time at which scan information is received is at least an hour after the capturing diagnostic medical images.

6. The method of claim 3, further comprising presenting, on a display, visual guidance to the target site based on a current position of an ultrasound probe.

7. The method of claim 1, wherein the architectural map of the breast tissue includes a margin of normal tissue around the target site.

8. The method of claim 1, wherein the diagnostic medical images of breast tissue are captured using ultrasound.

9. The method of claim 8, wherein the ultrasound performs B-Mode imaging.

10. The method of claim 8, wherein the images of breast tissue are also captured using digital breast tomosynthesis.

11. The method of claim 1, wherein the diagnostic medical images of breast tissue are captured using magnetic resonance imaging (MRI).

12. The method of claim 1, further comprising recording location coordinates of the target site.

13. The method of claim 12, wherein the location coordinates of the target site are defined by a clock position relative to a nipple of the breast, a depth from a surface of the breast, and a distance from the nipple.

14. The method of claim 13, wherein vascularity is measured using Doppler imaging.

15. The method of claim 14, wherein the Doppler imaging is microflow Doppler imaging.

16. The method of claim 1, wherein stiffness is measured using elastography.

17. The method of claim 16, wherein the elastography is shear-wave elastography.

18. A system for mapping a region of interest within a breast, the system comprising:
at least one data store;
a processor; and
a memory storing instructions that, when executed by the processor,
facilitate performance of operations, comprising:
mapping the region of interest within the breast by:
recording at least one image of the region of interest using diagnostic medical imaging;
measuring vascularity of the region of interest; and
measuring density of the region of interest; and
saving the at least one image, the set of coordinates, the vascularity, and the density as an architectural map in an electronic record associated with the breast.

19. The system of claim 18, wherein the operations further comprise:
at a later time, receiving scan information obtained from imaging of the breast;
accessing the architectural map from the electronic record associated with the breast; and
analyzing the scan information to identify the region of interest based on the architectural map.

20. The system of claim 19, further comprising a tracking system configured to:
receive a current position and orientation of an ultrasound probe from a probe localization transceiver of the ultrasound probe; and
display the current position and orientation of the ultrasound probe relative to the breast on a graphical user interface including images of the breast that are obtained during a scan of the breast.

21. The system of claim 19, further comprising a tracking system configured to:
determine a current position and orientation of an ultrasound probe based on images captured by a camera system; and
display the current position and orientation of the ultrasound probe relative to the breast on a graphical user interface including images of the breast that are obtained during a scan of the breast.

22. The system of claim 18, wherein the diagnostic medical imaging is ultrasound imaging and x-ray imaging.

23. The system of claim 18, further comprising recording a set of coordinates indicating a location of the region of interest and saving the set of coordinates with the architectural map.

24. The system of claim 18, wherein the vascularity and the density are measured using ultrasound.

25. The system of claim 24, wherein the ultrasound performs B-Mode imaging.

26. The system of claim 22, wherein the x-ray imaging is performed using digital breast tomosynthesis.

27. A non-transitory machine-readable storage medium, comprising executable instructions that, when executed by a processor, facilitate performance of operations, comprising:
capturing ultrasound images of an entire breast;
recording location coordinates of a region of interest within breast tissue of the breast;
measuring, using microflow Doppler, vascularity of the breast tissue including the region of interest;
measuring, using shear-wave elastography, stiffness of the breast tissue including the region of interest;
saving the images, the location coordinates, the vascularity, and the stiffness as an architectural map in an electronic record associated with the breast;
at a later time, receiving scan information obtained from imaging of the breast;
accessing the architectural map from the electronic record associated with the breast; and
analyzing the scan information to identify the region of interest based on the architectural map.

28. The non-transitory machine-readable storage medium of claim 27, wherein the region of interest is a biopsy site.

29. The non-transitory machine-readable storage medium of claim 27, wherein the later time is at least one day after the ultrasound images of the entire breast are captured, and wherein the scan information obtained from imaging of the breast is analyzed in comparison to the architectural map to determine if any changes to the breast tissue have occurred.
